(19) United States
(12) Patent Application Publication
(10) Pub. No.: US 2026/0055467 A1
(43) Pub. Date: Feb. 26, 2026

(54) COMPOSITIONS AND METHODS FOR DETECTION AND TREATMENT OF CANINE CANCERS

(71) Applicant: THE TRANSLATIONAL GENOMICS RESEARCH INSTITUTE, Phoenix, AZ (US)

(72) Inventors: Puiyee Agnes Chan, Phoenix, AZ (US); Yongwook Choi, Phoenix, AZ (US); Nicholas Schork, Phoenix, AZ (US)

(21) Appl. No.: 18/875,116

(22) PCT Filed: Jun. 19, 2023
(86) PCT No.: PCT/US2023/068684

§ 371 Date: Dec. 13, 2024

Related U.S. Application Data
(60) Provisional application No. 63/353,546, filed on Jun. 17, 2022.

(51) Int. Cl. C12Q 1/6886

(52) U.S. Cl. CPC C12Q 1/6886; C12Q 2600/158

(57) ABSTRACT

The present disclosure relates to methods of selecting and/or treating a subject for treatment of a cancer with a G4-stabilizing ligand based on expression of oncogene with a G4 motif comprising GₓN₁₋₇GₓN₁₋₇GₓN₁₋₇GₓN₁₋₇ (SEQ ID NO: 15) where N refers to any base and x≥3 in the subject. The disclosure also provides methods of detecting a cancer cell susceptible to growth inhibition with a G4-stabilizing ligand and methods of treating a cancer in a subject with a G4-stabilizing ligand.

### CROSS-REFERENCE TO RELATED APPLICATIONS

This disclosure claims priority to and the benefit of U.S. Provisional Application No. 63/353,546, filed Jun. 17, 2022, entitled “Compositions and Methods for Detection and Treatment of Canine Cancers.” The benefit under 35 USC § 119(e) of the United States Provisional Application is hereby claimed, and the entire disclosure of the aforementioned application is hereby incorporated herein by reference.

### ACKNOWLEDGMENT OF GOVERNMENT SUPPORT

This invention was made with governmental support under award numbers AG064706 and AG023122 awarded by the National Institutes of Health (NIH). The United States government has certain rights in the invention.

### INCORPORATION-BY-REFERENCE OF MATERIAL ELECTRONICALLY FILED

The official copy of the sequence listing is submitted electronically in ST.26 XML format having the file name “91482-253WO-PCT_SeqList” created on Jun. 19, 2023, and having a size of 38,810 bytes, and is filed concurrently with the specification. The Sequence Listing ST.26 XML file is part of the specification and is herein incorporated by reference in its entirety.

### TECHNICAL FIELD

The present invention is related to methods for diagnosing, treating, and preventing various canine cancers.

### BACKGROUND

Pets play an important role in many people's lives, and consequently many pet owners will go to considerable lengths to treat their pets for major illnesses including cancer. Cancer is one of the major forms of mortality in pets, and therefore the pet owners desire ways of treating this disease in their pets to increase their longevity.

The incidence of cancer in companion animals such as dogs is increasing, and cancer is now considered to be the leading cause of death in older animals. It is believed that the annual incidence rate for cancers in dogs is about 2 to 2.5%.

The cancers having the highest incidence in dogs are lymphoma (about 20%); mast cell tumor (about 18%); soft tissue sarcoma (about 10%); hemangiosarcoma (about 10%); osteosarcoma (about 9%). The remaining segments typically include squamous cell carcinoma, mammary carcinoma, melanoma, histiocytoma and fibrosarcoma.

Approaches to cancer treatment in veterinary oncology include surgery, radiation therapy, hyperthermia therapy, photodynamic therapy and chemotherapy. Gene therapy and immunotherapy have not been widely implemented.

Present methods of treating cancer in pets focus primarily on surgical resection of solid tumors. Surgery is expensive, and moreover, is not suitable treatment for many cancers. Among these are leukemias and lymphomas, where surgery obviously is not an option, but this class includes highly disseminated malignancies as well as ones with poorly defined margins or those arising in inoperable locations.

Treatment for cancer is improving with an ever-growing range of available drugs and radiation treatments even though the diagnosis of the cancer type and location and subsequently the treatment and the monitoring have to be carefully devised on a patient-by-patient basis, given the large number of different types of cancer. However, one common aspect that significantly improves the success rate of treatment is early detection. For most cancer types, early detection improves the outcome of the treatment and the management of the disease.

Since early detection of cancer plays such a crucial role in the success of the treatment (e.g., survival rate of patients), the screening of some cancers (e.g., breast cancer, colon cancer) may be recommended even in subjects appearing healthy and without showing symptoms that may be attributed to cancer. However, screening is typically a medical (or veterinary) procedure that may be invasive (e.g., requires biopsies) and is generally too expensive to make it a routine procedure in non-suspected patients. Additionally, because of the cost a practitioner may be reluctant to order a full screening for cancer when a patient is showing only mild symptoms or a few symptoms that would indicate cancer.

Therefore, there is a need for screening methods for cancer in companion animals that are cost effective and less invasive, allowing a practitioner to detect cancer at an early stage and pursue other methods of diagnosis and treatment. The same methods may be used to monitor subjects during and after treatment cost-effectively. Such methods of early detection along with more effective treatments would greatly benefit companion animals and increase their longevity.

### SUMMARY

The present disclosure is directed to a method of selecting a subject for treatment of a cancer with a G4-stabilizing ligand. The method comprises two main steps: identifying an oncogene expressed in the genome of the subject with a G4 motif comprising GₓN₁₋₇GₓN₁₋₇GₓN₁₋₇ GₓN₁₋₇ (SEQ ID NO: 15) where N refers to any base and x≥3; and determining an expression level of the oncogene in the subject, wherein an increase in the level of expression of at least 50% higher than that of non-cancerous cells that are adjacent to the cancer identifies the subject for treatment with the G4-stabilizing ligand.

In certain aspects, the expression level of the oncogene is determined at the protein level by Western blotting, ELISA-based detection, in situ immunohistochemistry, in situ immunocytochemistry, affinity chromatography, or an enzyme immunoassay.

In other aspects, the expression level of the oncogene is determined at the nucleic acid level by Northern blotting, real time PCR, RT PCR, quantitative PCR, RT-qPCR, a hybridization array, branched nucleic acid amplification, RNA-seq, in situ hybridization, amplification followed by HPLC detection or MALDI-TOF mass spectrometry, or next generation sequencing (NGS).

In one aspect, the method further comprises administering the G4-stabilizing ligand to the subject. In another aspect, the oncogene is MYCN.

In some aspects, the cancer is selected from the group consisting of glioma, colorectal cancer (CRC), bladder cancer, kidney cancer, non-small cell lung cancer (NSCLC), neuroblastoma, multiple myeloma, T-cell chronic lymphocytic leukemia (T-CLL), acute lymphocytic leukemia (ALL), acute myeloid leukemia (AML), ovarian cancer, uterine cancer, melanoma, and glioblastoma multiforme. In one aspect, the cancer is selected from the group consisting of ovarian cancer, uterine cancer, melanoma, and glioblastoma multiforme.

In certain aspects, the G4-stabilizing ligand is selected from the group consisting of CX-3543, CX-5461, telomestatin, BMSG-SH-3, MM41, BRACO-19, quarfloxin, CM03, PDP, and pharmaceutically acceptable salts thereof.

In other aspects, the method further comprises administering to the subject a chemotherapeutic agent selected from the group consisting of an alkylating agent, plant alkaloid, antimetabolite, antitumor antibiotic, platinum compound, topoisomerase I inhibitor, retinoid, and combinations thereof.

In one aspect, the subject is a canine. In another aspect, the subject is a feline.

In yet other aspects, the present disclosure provides a method of detecting one or more cancer cells susceptible to growth inhibition with a G4-stabilizing ligand, the method comprising: identifying an oncogene in the genome of the one or more cancer cells with a G4 motif comprising GₓN₁₋₇GₓN₁₋₇GₓN₁₋₇GₓN₁₋₇ (SEQ ID NO: 15) where N refers to any base and x≥3; determining an expression level of the oncogene in the cancer cells, wherein an increase in the level of expression of the oncogene of at least 50% higher than that of non-cancerous cells that are adjacent to the cancer cells identifies the susceptibility of the one or more cancer cells to growth inhibition with the G4-stabilizing ligand.

In some aspects, the method further comprises administering a G4-stabilizing ligand to the cancer cell to confirm susceptibility to growth inhibition. In certain aspects, the method is an in vitro method.

In yet other aspects, the present disclosure provides a method of treating a cancer in a subject, the method comprising: identifying an oncogene expressed in the genome of the subject with a G4 motif comprising GₓN₁₋₇GₓN₁₋₇GₓN₁₋₇GₓN₁₋₇ (SEQ ID NO: 15) where N refers to any base and x≥3; determining an expression level of the oncogene in the subject, wherein an increase in the level of expression of the oncogene of at least 50% higher than that of non-cancerous cells that are adjacent to the cancer identifies the subject for treatment with a G4-stabilizing ligand; and administering a G4-stabilizing ligand to the subject.

In one aspect, the oncogene is MYCN. In another aspect, the G4-stabilizing ligand is selected from the group consisting of CX-3543, CX-5461, telomestatin, BMSG-SH-3, MM41, BRACO-19, quarfloxin, CM03, PDP, and pharmaceutically acceptable salts thereof.

The foregoing features and elements may be combined in various combinations without exclusivity, unless expressly indicated otherwise. These features and elements as well as the operation thereof will become more apparent in light of the following description. It should be understood, however, that the following description is intended to be exemplary in nature and non-limiting.

### BRIEF DESCRIPTION OF THE FIGURES

Illustrative and exemplary embodiments of the invention are shown in the drawings in which:

FIG. 1A depicts the results of an analysis of orthologous groups (OGs) present in several canine genome assemblies along with their corresponding human orthologs identified in the Catalogue of Somatic Mutations in Cancer (COSMIC) Census. FIG. 1B depicts an analysis of one of the OGs, MYCN, comparing an older canine genome assembly (i.e., CanFam3.1) with several newer genome assemblies of higher quality (i.e., Dog10K). This comparison indicates an N-terminal truncation present in the MYCN sequence from CanFam3.1. FIG. 1C depicts additional information relevant to the analysis presented in FIG. 1B including sequence identity information for each of the canine genome assemblies.

FIG. 2 depicts a 1.2 kb gap present in the MYCN gene sequenced in the canFam3.1 canine genome assembly and two other canine whole genome sequencing (WGS) samples. Comparison of this 1.2 kb gap with the MYCN gene sequence present in the high-quality Dog10K reference genome assembly indicates a highly G-rich region typical of those containing G4 structures.

FIG. 3 depicts the expression of GₓN₁₋₇GₓN₁₋₇GₓN₁₋₇GₓN₁₋₇ (SEQ ID NO: 15) where N refers to any base including guanine and x≥3 used to identify the G4 motif (see Jean-Michel Garant, J. et al., Database, Volume 2015, 2015, bav059).

FIG. 4 depicts the relatively high incidence of G4 structures associated with oncogenes. See Ghosh, A. et al., (2021) Nucleic acids research 49 (4): 2333-2345.

FIG. 5A depicts mutations in the human MYCN gene with P44L/H identified as a cancer hot spot. Alignment of the human and canine MYCN genes shows that the 1.2 kb gap region identified in the CanFam3.1 genome assembly spans the P44L/H cancer hotspot. FIGS. 5B and 5C depict additional information related to the P44L/H cancer hotspot including its presence in ovarian cancer, uterine cancer, melanoma, and glioblastoma multiforme.

FIG. 6 depicts several predicted G4 motifs along the canine MYCN gene. Importantly, a predicted G4 motif is found within the 1.2 kb gap G-rich region which is missing from the CanFam3.1 assembly.

### DETAILED DESCRIPTION

It is to be understood that unless specifically stated otherwise, references to “a,” “an,” and/or “the” may include one, or more than one, and that reference to an item in the singular may also include the item in the plural. Reference to an element by the indefinite article “a,” “an” and/or “the” does not exclude the possibility that more than one of the elements are present, unless the context clearly requires that there is one and only one of the elements.

As used herein, the term “comprise,” and conjugations or any other variation thereof, are used in its non-limiting sense to mean that items following the word are included, but items not specifically mentioned are not excluded.

A sample, cell, tumor, or cancer which “expresses” one or more oncogenes at an increased expression level relative to a median level of expression (e.g., the median level of expression of the one or more oncogenes in the type of cancer (or in a cancer type, wherein the “cancer type” is meant to include cancerous cells (e.g., tumor cells, tumor tissues) and/or non-cancerous cells (e.g., stromal cells, stromal tissues) that surround the cancerous/tumor environment) is one in which the expression level of one or more oncogenes is considered to be a “high oncogene expression level” to a skilled person for that type of cancer. Generally, such a level will be in the range from about 50% up to about 100% or more (e.g., 50%, 55%, 60%, 65%, 70%, 75%, 80%, 85%, 90%, 95%, 100%, or more) relative to oncogene levels in a population of samples, cells, tumors, or cancers of the same cancer type. For instance, the population that is used to arrive at the median expression level may be particular sets of cancer samples (e.g., bladder cancer, breast cancer, colorectal cancer, gastric cancer, liver cancer, melanoma, lung cancer (e.g., non-small cell lung carcinoma), ovarian cancer, or renal cell carcinoma) generally, or subgroupings thereof, such as chemotherapy-resistant cancer, platinum-resistant cancer, as well as advanced, refractory, or recurrent cancer samples.

In some embodiments, the oncogene level of expression that indicates the need for treatment is at least 40% higher than that of non-cancerous cells that are adjacent to the cancer cells. In certain embodiments, the level of expression is 40%, 50%, 55%, 60%, 65%, 70%, 75%, 80%, 85%, 90%, 95%, 100%, or more than the non-cancerous cells that are adjacent to the cancer cells.

“High/increased level of expression” means increased relative to controls or normals using accepted statistical criteria. In this light, an increase in the level of expression of the oncogene relative to a median level identifies the subject for treatment with the G4-stabilizing ligand. Additionally, an increase in the level of expression of the oncogene relative to a control or normal gene expression level using accepted statistical analysis criteria identifies the subject for treatment with the G4-stabilizing ligand.

For example, accepted statistical criteria can be simple t-tests of the difference in expression between the target sample and the ‘controls’ (e.g., made up of a number of normal samples, or a number of samples taken from normal tissue from the target patient) or what is referred to as ‘fold-change’ which is simply the ratio of target sample expression to control sample expression (even if just one control sample). In some embodiments, that criteria can be ‘based on x-fold change of expression’ where ‘x’ is defined by the setting (e.g., 2 or 3 fold change would mean that the target sample expression is 2 or 3 times higher than controls).

The term “adjacent” means next to or adjoining something else.

By “determining the expression level” used in reference to a particular biomarker (e.g., one or more oncogenes), means expression of the biomarker(s) (e.g., one or more oncogenes) in a cancer-associated biological environment (e.g., expression of the biomarker(s) in the tumor cells), tumor-associated cells (e.g., tumor-associated stromal cells), as determined using a diagnostic test, any of the detection methods described herein, or the similar.

As used herein, the term “cancer” refers to a malignant tumor that has undergone characteristic anaplasia with loss of differentiation, increase rate of growth, invasion of surrounding tissue, and is capable of metastasis. For example, thyroid cancer is a malignant tumor that arises in or from thyroid tissue, and breast cancer is a malignant tumor that arises in or from breast tissue (such as a ductal carcinoma). Residual cancer is cancer that remains in a subject after any form of treatment given to the subject to reduce or eradicate the cancer. Metastatic cancer is a tumor at one or more sites in the body other than the site of origin of the original (primary) cancer from which the metastatic cancer is derived. Cancer includes, but is not limited to, solid tumors.

As used herein, the term “tumor” refers to an abnormal growth of cells, which can be benign or malignant. Cancer is a malignant tumor, which is characterized by abnormal or uncontrolled cell growth. Other features often associated with malignancy include metastasis, interference with the normal functioning of neighboring cells, release of cytokines or other secretory products at abnormal levels and suppression or aggravation of inflammatory or immunological response, invasion of surrounding or distant tissues or organs, such as lymph nodes, etc. “Metastatic disease” refers to cancer cells that have left the original tumor site and migrate to other parts of the body for example via the bloodstream or lymph system.

The amount of a tumor in an individual is the “tumor burden” which can be measured as the number, volume, or weight of the tumor. A tumor that does not metastasize is referred to as “benign.” A tumor that invades the surrounding tissue and/or can metastasize is referred to as “malignant.” Examples of hematological tumors include leukemias, including acute leukemias (such as 11q23-positive acute leukemia, acute lymphocytic leukemia, acute myelocytic leukemia, acute myelogenous leukemia and myeloblastic, promyelocytic, myelomonocytic, monocytic and erythroleukemia), chronic leukemias (such as chronic myelocytic (granulocytic) leukemia, chronic myelogenous leukemia, and chronic lymphocytic leukemia), polycythemia vera, lymphoma, Hodgkin's disease, non-Hodgkin's lymphoma (indolent and high grade forms), multiple myeloma, Waldenstrom's macroglobulinemia, heavy chain disease, myelodysplastic syndrome, hairy cell leukemia and myelodysplasia.

Examples of solid tumors, such as sarcomas and carcinomas, include fibrosarcoma, myxosarcoma, liposarcoma, chondrosarcoma, osteogenic sarcoma, and other sarcomas, synovioma, mesothelioma, Ewing's tumor, leiomyosarcoma, rhabdomyosarcoma, colon carcinoma, lymphoid malignancy, pancreatic cancer, breast cancer (including basal breast carcinoma, ductal carcinoma and lobular breast carcinoma), lung cancers, ovarian cancer, prostate cancer, hepatocellular carcinoma, squamous cell carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, seminoma, bladder carcinoma, and CNS tumors (such as a glioma, astrocytoma, medulloblastoma, craniopharyngioma, ependymoma, pinealoma, hemangioblastoma, acoustic neuroma, oligodendroglioma, meningioma, melanoma, neuroblastoma and retinoblastoma). In several examples, a tumor is melanoma, lung cancer, lymphoma breast cancer or colon cancer.

In some aspects, the cancer is selected from the group consisting of leukemia, non-small cell lung cancer, colon cancer, CNS cancer, melanoma, ovarian cancer, renal cancer, prostate cancer, breast cancer, and multiple myeloma.

The sample in this method is preferably a biological sample from a subject. The term “sample” or “biological sample” is used in its broadest sense. Depending upon the embodiment of the invention, for example, a sample may comprise a bodily fluid including whole blood, serum, plasma, urine, saliva, cerebral spinal fluid, semen, vaginal fluid, pulmonary fluid, tears, perspiration, mucus and the like; an extract from a cell, chromosome, organelle, or membrane isolated from a cell; a cell; genomic DNA, RNA, or cDNA, in solution or bound to a substrate; a tissue; a tissue print, or any other material isolated in whole or in part from a living subject or organism. Biological samples may also include sections of tissues such as biopsy and autopsy samples, and frozen sections taken for histologic purposes such as blood, plasma, serum, sputum, stool, tears, mucus, hair, skin, and the like. Biological samples also include explants and primary and/or transformed cell cultures derived from patient tissues.

The terms “peptide,” “polypeptide,” and “protein” are used interchangeably herein to refer to polymers of amino acids of any length, chemically or biochemically modified or derivatized amino acids, and polypeptides having modified peptide backbones. These terms also include proteins that are post-translationally modified through reactions that include glycosylation, acetylation and phosphorylation. The term “at least a portion” of a polypeptide means a portion having the minimal size characteristics of such sequences, or any larger fragment of the full-length molecule, up to and including the full-length molecule. For example, a portion of a polypeptide may be 4 to 15 amino acids, or may be 4 amino acids, 5 amino acids, 6 amino acids, 7 amino acids, and so on, up to a full-length polypeptide. A portion of a polypeptide useful as an epitope may be as short as 4 amino acids. A portion of a polypeptide that performs the function of the full-length polypeptide would generally be longer than 4 amino acids.

The term “amino acid” refers to naturally occurring and synthetic amino acids, as well as amino acid analogs and amino acid mimetics that function in a manner that is similar to the function of naturally occurring amino acids. Naturally occurring amino acids are those encoded by the genetic code, as well as those amino acids that are later modified. Unnatural amino acids are not encoded by the genetic code and can, but do not necessarily have the same basic structure as a naturally occurring amino acid. “Amino acid analogs” refers to compounds that have the same basic chemical structure as a naturally occurring amino acid, i.e., a carbon that is bound to a hydrogen, a carboxyl group, an amino group, and an R group, e.g., homoserine, norleucine, methionine sulfoxide, methionine methyl sulfonium. Such analogs may have modified R groups (e.g., norleucine) or modified peptide backbones, but retain the same basic chemical structure as a naturally occurring amino acid. “Amino acid mimetics” refers to chemical compounds that have a structure that is different from the general chemical structure of an amino acid, but that functions in a manner similar to a naturally occurring amino acid.

Amino acids may be referred to by either the three letter symbols or by the one-letter symbols recommended by the IUPAC, the IUAPC letter code are as follows: G=Glycine; A=Alanine; L=Leucine; M=Methionine; F=Phenylalanine; W=Tryptophan; K=Lysine; Q=Glutamine; E=Glutamic Acid; S=Serine; P=Proline; V=Valine; I=Isoleucine; C=Cysteine; Y=Tyrosine; H=Histidine; R=Arginine; N=Asparagine; D=Aspartic Acid; T=Threonine.

The terms “homologous” and “similar” refer to the relationship between proteins that possess a “common evolutionary origin,” including proteins from superfamilies (e.g., the immunoglobulin superfamily) and homologous proteins from different species. Such proteins (and their encoding genes) have sequence homology, as reflected by their sequence similarity, whether in terms of percent similarity or the presence of specific residues or motifs as conserved positions. In a specific embodiment, two peptide sequences are “substantially homologous or similar” when at least about 80%, or at least about 90%, or at least about 95) of the amino acids match over the defined lengths of the amino acid sequences.

The term “variant” applies to both amino acid and nucleic acid sequences. Because of the degeneracy of the genetic code, a large number of functionally identical nucleic acids encode any given protein. For instance, the codons GCA, GCC, GCG and GCU all encode the amino acid alanine. Variants may include individual substitutions, deletions or additions to a nucleic acid, peptide, polypeptide, or protein sequence which alters, adds, or deletes a single amino acid or a small percentage of amino acids in the encoded sequence.

“Function-conservative variants” are those in which a given amino acid residue in a protein or enzyme has been changed without altering the overall conformation and function of the polypeptide, including, but not limited to, replacement of an amino acid with one having similar properties (such as, for example, polarity, hydrogen bonding potential, acidic, basic, hydrophobic, aromatic, and the like). Amino acids with similar properties are well known in the art. For example, arginine, histidine and lysine are hydrophilic-basic amino acids and may be interchangeable. Similarly, isoleucine, a hydrophobic amino acid, may be replaced with leucine, methionine or valine. Such changes are expected to have little or no effect on the apparent molecular weight or isoelectric point of the protein or polypeptide.

Amino acids other than those indicated as conserved may differ in a protein so that the percent protein or amino acid sequence similarity between any two proteins of similar function may vary and may be, for example, from 70% to 99% as determined according to an alignment scheme. A “variant” also includes a polypeptide which has at least 60% amino acid identity as determined by BLAST or FASTA algorithms, preferably at least 75% most preferably at least 85%, and even more preferably at least 90%, and still more preferably at least 95%, and which has the same or substantially similar properties or functions as the native or parent protein to which it is compared. A particular variant is a “gain-of-function” variant, meaning a polypeptide variant in which the change of at least one given amino acid residue in a protein or enzyme modifies—often increasing—a specific function of the polypeptide, including, but not limited to protein activity. The change in amino acid residue can be replacement of an amino acid with one having similar properties.

A target or a marker may be any molecular structure produced by a cell, expressed inside the cell, accessible on the cell surface, or secreted by the cell. A marker may be any protein, carbohydrate, fat, nucleic acid, catalytic site, or any target of these such as an enzyme, glycoprotein, cell membrane, virus, cell, organ, organelle, or any uni- or multimolecular structure or any other such structure now known or yet to be disclosed whether alone or in combination. A target may also be called a marker and the terms are used interchangeably.

A target may be represented by the sequence of amino acids, or sequence of one or more strands of a nucleic acid from which it may be derived. For example, a target may be represented by a protein sequence. Alternatively, a target may be represented by a nucleic acid sequence, the protein or peptide or the fragments thereof encoded by the nucleic acid sequence. Examples of such nucleic acids include both single stranded and double stranded nucleic acid sequences including miRNA, tRNA, siRNA, mRNA, cDNA, or genomic DNA sequences including complimentary sequences. The concept of a marker is not limited to the products of the exact nucleic acid sequence or protein sequence by which it may be represented. Rather, a marker encompasses all molecules that may be detected by a method of assessing the expression of the marker. Examples of molecules encompassed by a marker include point mutations, silent mutations, deletions, frameshift mutations, translocations, alternative splicing derivatives, differentially methylated sequences, differentially modified protein sequences, truncations, soluble forms of cell membrane associated markers, and any other variation that results in a product that may be identified as the marker. The term “target” further encompasses the products (i.e., proteins) of the gene or a gene allele thereof, whose expression or activity is directly or indirectly associated with a particular phenotype or cellular condition, or physiological characteristic.

Indirect methods of detecting a marker generally involve assessing the expression of material created from a genomic DNA template such as an RNA or protein molecule. Such expression may be assessed by any of a number of methods used currently in the art and yet to be developed. Examples include any nucleic acid detection method including the following nonlimiting examples, microarray RNA analysis, RNA in situ hybridization, RNAse protection assay, Northern blot, reverse transcription PCR, and quantitative reverse transcription PCR. Other examples include any process of detecting expression that uses an antibody including the following nonlimiting examples, flow cytometry, immunohistochemistry, ELISA, Western blot, Northwestern blot, and immunoaffinity chromatography. Antibodies may be monoclonal, polyclonal, or any antibody fragment including a Fab, F(ab)₂, Fv, scFv, phage display antibody, peptibody, multispecific ligand, or any other reagent with specific binding to a target. Other methods of assessing protein expression include the following nonlimiting examples: HPLC, mass spectrometry, protein microarray analysis, PAGE analysis, isoelectric focusing, 2-D gel electrophoresis, and enzymatic assays.

Agents that interact with a therapeutic target to result in a desirable therapeutic effect may include a pharmaceutically active ingredient or pharmaceutically acceptable salt thereof, a drug, a toxin, a chemical, a small organic molecule, a large molecule or peptide or an antibody. Large-molecule pharmaceuticals refer to pharmaceutical agents having a molecular weight greater than about 1000 Daltons, e.g., peptidic drugs, vaccines and hormones. The term “antibody” is used herein in the broadest sense and refers generally to a molecule that contains at least one antigen binding site that immunospecifically binds to a particular antigen target of interest. Antibody thus includes, but is not limited to, native antibodies and variants thereof, fragments of native antibodies and variants thereof, peptibodies and variants thereof, and antibody mimetics that mimic the structure and/or function of an antibody or a specified fragment or portion thereof, including single chain antibodies and fragments thereof. The term, thus, includes full length antibodies and/or their variants as well as immunologically active fragments thereof, thus encompassing, antibody fragments capable of binding to a biological molecule (such as an antigen or receptor) or portions thereof, including, but not being limited to, Fab, Fab′, F(ab′)2, facb, pFc′, Fd, Fv or scFv (See, e.g., CURRENT PROTOCOLS IN IMMUNOLOGY, (Colligan et al., eds., John Wiley & Sons, Inc., NY, 1994-2001).

In the preparation of the pharmaceutical compositions described in the teachings herein, a variety of vehicles and excipients and routes of administration may be used, as will be apparent to the skilled artisan. Representative formulation technology is taught in, inter alia, Remington: The Science and Practice of Pharmacy, 19th Ed., Mack Publishing Co., Easton, Pa. (1995) and Handbook of Pharmaceutical Excipients, 3rd Ed, Kibbe, A. H. ed., Washington D.C., American Pharmaceutical Association (2000); hereby incorporated by reference in their entirety.

Pharmaceutically acceptable salts of the presently disclosed compounds also include those formed from cations such as sodium, potassium, aluminum, calcium, lithium, magnesium, zinc, and from bases such as ammonia, ethylenediamine, N-methyl-glutamine, lysine, arginine, ornithine, choline, N,N′-dibenzylethylenediamine, chloroprocaine, diethanolamine, procaine, N-benzylphenethylamine, diethylamine, piperazine, tris(hydroxymethyl)aminomethane, and tetramethylammonium hydroxide. These salts may be prepared by standard procedures, for example by reacting the free acid with a suitable organic or inorganic base. Any chemical compound recited in this specification may alternatively be administered as a pharmaceutically acceptable salt thereof. “Pharmaceutically acceptable salts” are also inclusive of the free acid, base, and zwitterionic forms. Descriptions of suitable pharmaceutically acceptable salts can be found in Handbook of Pharmaceutical Salts, Properties, Selection and Use, Wiley VCH (2002). When compounds disclosed herein include an acidic function such as a carboxy group, then suitable pharmaceutically acceptable cation pairs for the carboxy group are well known to those skilled in the art and include alkaline, alkaline earth, ammonium, quaternary ammonium cations and the like. Such salts are known to those of skill in the art. For additional examples of pharmacologically acceptable salts, see Berge et al., J. Pharm. Sci. 66:1 (1977).

In other embodiments there is provided a pharmaceutical composition as described herein together with a pharmaceutically acceptable carrier, diluent or excipient. As used herein, “carrier(s)” can be used interchangeably with “excipient(s).” Carriers include any substance that may be administered with the one or more disclosed compounds with the intended purpose of facilitating, assisting, or helping the administration or other delivery of the compound. Carriers include any liquid, solid, semisolid, gel, aerosol or anything else that may be combined with the disclosed compound to aid in its administration. Examples include diluents, adjuvants, excipients, water, and oils (including petroleum, animal, vegetable or synthetic oils). The pharmaceutical composition may be formulated as powders, granules, solutions, suspensions, aerosols, solids, pills, tablets, capsules, gels, topical creams, suppositories, transdermal patches, and other formulations known in the art.

The pharmaceutical compositions described herein may be administered by any means that enables the active agent to reach the agent's site of action in the body of the subject. The dosage administered varies depending upon factors, such as: pharmacodynamic characteristics; mode and route of administration; age, health, and weight of the recipient subject; nature and extent of symptoms; concurrent treatments; and frequency of treatment.

As used herein, the terms “administration” and “administering” of an agent to a subject include any route of introducing or delivering the agent to a subject to perform its intended function. Administration can be carried out by any suitable route, including intravenously, intramuscularly, intraperitoneally, inhalationally, intranasally, or subcutaneously. Administration includes self-administration and the administration by another.

The term “effective amount” or “therapeutically effective amount” refers to that amount of an agent or combination of agents as described herein that is sufficient to affect the intended application including, but not limited to, disease treatment and/or disease prevention. A therapeutically effective amount may vary depending upon the intended application (in vitro or in vivo), or the subject and disease condition being treated (e.g., the weight, age and gender of the subject), the severity of the disease condition, or the manner of administration. The term also applies to a dose that will induce a particular response in target cells. The specific dose will vary depending on the particular agents chosen, the dosing regimen to be followed, whether the agent is administered in combination with other agents, timing of administration, the tissue to which it is administered, and the physical delivery system in which the compound is carried.

The terms “treatment,” “treating,” “treat,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment”, as used herein, covers any treatment of a disease in a mammal, particularly in a human, and includes: (a) preventing the disease from occurring in a subject which may be predisposed to the disease but has not yet been diagnosed as having it; (b) inhibiting the disease, i.e., arresting its development or progression; and (c) relieving the disease, i.e., causing regression of the disease and/or relieving one or more disease symptoms. “Treatment” is also meant to encompass delivery of an agent in order to provide for a pharmacologic effect, even in the absence of a disease or condition. For example, “treatment” encompasses delivery of a composition that can elicit an immune response or confer immunity in the absence of a disease condition, e.g., in the case of a vaccine.

As used herein, the term “patient” or “subject” refers to any organism to which a provided composition is or may be administered, e.g., for experimental, diagnostic, prophylactic, cosmetic, and/or therapeutic purposes. For example, subject may refer to a human or a non-human animal. In some aspects, subject refers to any vertebrate including, without limitation, humans and other primates (e.g., chimpanzees and other apes and monkey species), farm animals (e.g., cattle, sheep, pigs, goats and horses), domestic mammals (e.g., dogs and cats), laboratory animals (e.g., rodents such as mice, rats, and guinea pigs), and birds (e.g., domestic, wild and game birds such as chickens, turkeys and other gallinaceous birds, ducks, geese, and the like). In some embodiments, the subject is a mammal. In further embodiments, the subject is a canine.

The term “G-quadruplex” as used herein refers to a four-stranded helical nucleic acid structure comprising multiple stacked G-tetrads, each of which consists of four guanine bases or chemical analogues of guanine that associate in a cyclical manner through Hoogsteen hydrogen bonds and may be further stabilized through coordination to a cation in the center. The body of stacked G-tetrads, comprising a total of 2 or more layers, is collectively referred to as the G-tetrad core. Each of the four guanine columns constituting the G-tetrad core can arise from a single (continuous column) or two (discontinuous column) separate guanine stretch/es. The G-quadruplex structure is thus a robust four-stranded helical structure spontaneously formed by G-rich oligonucleotide sequences under physiological conditions. Said G-quadruplex structure has two major structural components, a four-stranded G-tetrad core and three intervening loops.

G-quadruplex structures are highly diverse with regards to their relative strand orientations and loop types, resulting in different topologies including: (a) parallel-type in which four strands point in the same direction; (b) hybrid “3+1” type in which three strands point in one direction and the fourth strand points in the opposite one; (c) antiparallel-type in which two strands point in one direction and two strands point in the opposite direction. These topologies lead to different structural molecular shapes, with various loops and grooves of different size and accessibility (Phan A. T., FEBS J. 2010, 277, 1107). The structural polymorphism of G-quadruplexes depends on their nucleotide sequences and the environmental conditions.

In various embodiments, the G-quadruplex structure comprises: (a) a nucleic acid molecule comprising the nucleic acid sequence (g)_{w}(n)ₐ(g)ₓ((n)_{b}(g)_{y}(n)_{c}(g)_{z}, wherein w, x, y, z are independently of each other integers of at least 0; a, b, c are independently of each other integers of at least 0, and the sum of integers w, x, y, z is at least 8; (b) four nucleic acid molecules, wherein each of said molecules comprises a sequence of (g)_{z}, wherein z is an integer of at least 2; or (c) two nucleic acid molecules, wherein each of said molecules comprises a sequence of (g)_{y}(n)_{b}(g)_{z} wherein b is an integer of at least 0, y and z are independently of each other integers of at least 0, and the sum of integers y and z is at least 4. In various embodiments, the nucleic acid molecule comprises the nucleic acid sequence (g)_{w}(n)ₐ(g)ₓ((n)_{b}(g)_{y}(n)_{c}(g)_{z}, wherein w, x, y, z are independently of each other integers of at least 3 and a, b, c are independently of each other integers of at least 1. The term “g”, as used herein, relates to a guanine nucleobase or its chemical analogues. The term “n”, as used herein, relates to a nucleotide having a base that is selected from the group consisting of adenine, guanine, cytosine, uracil, and thymine, or their chemical analogues.

### Measurement of Expression Levels of an Oncogene

In some aspects, the present disclosure relates to the measurement of an expression level an oncogene. The expression level of an oncogene may be assessed by any method known in the art suitable for determination of specific protein levels in a patient sample and is preferably determined by an immunohistochemical (“IHC”) method employing antibodies specific for an oncogene. Such methods are well known and routinely implemented in the art, and corresponding commercial antibodies and/or kits are readily available. Preferably, the expression levels of the marker/indicator proteins of the invention are assessed using the reagents and/or protocol recommendations of the antibody or kit manufacturer. The skilled person will also be aware of further means for determining the expression level of an oncogene by IHC methods. Therefore, the expression level of one or more of the markers/indicators of the invention can be routinely and reproducibly determined by a person skilled in the art without undue burden. However, to ensure accurate and reproducible results, the invention also encompasses the testing of patient samples in a specialized laboratory that can ensure the validation of testing procedures.

Preferably, the expression level of an oncogene is assessed in a biological sample that contains or is suspected to contain cancer cells. The sample may be, for example, a tissue resection, a tissue biopsy, or a metastatic lesion obtained from a patient suffering from, suspected to suffer from, or diagnosed with cancer (e.g., bladder cancer, breast cancer, colorectal cancer, gastric cancer, liver cancer, melanoma, lung cancer (e.g., non-small cell lung carcinoma), ovarian cancer, or renal cell carcinoma). Preferably, the sample is a sample of a tissue, a resection or biopsy of a tumor, a known or suspected metastatic cancer lesion or section, or a blood sample, e.g., a peripheral blood sample, known or suspected to comprise circulating cancer cells. The sample may comprise both cancer cells, i.e., tumor cells, and non-cancerous cells, and, in certain embodiments, comprises both cancerous and non-cancerous cells. In aspects of the invention comprising the determination of gene expression in stroma components, the sample comprises both cancer/tumor cells and non-cancerous cells that are, e.g., associated with the cancer/tumor cells (e.g., tumor associated fibroblasts, endothelial cells, pericytes, the extra-cellular matrix, and/or various classes of leukocytes). In other aspects, the skilled artisan, e.g., a pathologist, can readily discern cancer cells from non-cancerous (e.g., stromal cells, endothelial cells, etc.). Methods of obtaining biological samples including tissue resections, biopsies, and body fluids, e.g., blood samples comprising cancer/tumor cells, are well known in the art. In some embodiments, the sample obtained from the patient is collected prior to beginning any treatment regimen or therapy, e.g., chemotherapy or radiation therapy for the treatment of cancer or the management or amelioration of a symptom thereof. Therefore, in some embodiments, the sample is collected before the administration of chemotherapeutic agents, or the start of chemotherapy or other treatment regimen (e.g., immunotherapy).

In addition to the methods described above, the invention also encompasses further immunohistochemical methods for assessing the expression level of one or more oncogenes, such as by Western blotting and ELISA-based detection. As is understood in the art, the expression level of the marker/indicator proteins of the invention may also be assessed at the mRNA level by any suitable method known in the art, such as Northern blotting, real time PCR, and RT PCR. Immunohistochemical- and mRNA-based detection methods and systems are outlined in textbooks, such as Lottspeich (Bioanalytik, Spektrum Akademisher Verlag, 1998) or Sambrook and Russell (Molecular Cloning: A Laboratory Manual, CSH Press, Cold Spring Harbor, N.Y., U.S.A., 2001). The described methods are of particular use for determining the expression levels of an oncogene in a patient or group of patients relative to control levels established in a population diagnosed with advanced stages of a cancer (e.g., bladder cancer, breast cancer, colorectal cancer, gastric cancer, liver cancer, melanoma, lung cancer (e.g., non-small cell lung carcinoma), ovarian cancer, or renal cell carcinoma).

For use in the detection methods described herein, the skilled person has the ability to label the polypeptides or oligonucleotides encompassed by the present invention. As routinely practiced in the art, hybridization probes for use in detecting mRNA levels and/or antibodies or antibody fragments for use in IHC methods can be labeled and visualized according to standard methods known in the art. Non-limiting examples of commonly used systems include the use of radiolabels, enzyme labels, fluorescent tags, biotin-avidin complexes, chemiluminescence, and the like.

The expression level of one or more of an oncogene can also be determined on the protein level by taking advantage of immunoagglutination, immunoprecipitation (e.g., immunodiffusion, immunoelectrophoresis, immune fixation), Western blotting techniques (e.g., in situ immunohistochemistry, in situ immunocytochemistry, affinity chromatography, enzyme immunoassays), and the like. Amounts of purified polypeptide may also be determined by physical methods, e.g., photometry. Methods of quantifying a particular polypeptide in a mixture usually rely on specific binding, e.g., of antibodies.

As mentioned above, the expression level of the marker/indicator proteins according to the present invention may also be reflected in increased or decreased expression of the corresponding oncogene. Therefore, a quantitative assessment of the gene product prior to translation (e.g., spliced, unspliced or partially spliced mRNA) can be performed in order to evaluate the expression of the corresponding oncogene(s). The person skilled in the art is aware of standard methods to be used in this context or may deduce these methods from standard textbooks (e.g., Sambrook, 2001). For example, quantitative data on the respective concentration/amounts of mRNA encoding one or more of an oncogene as described herein can be obtained by Northern Blot, Real Time PCR, and the like.

In certain aspects, the expression of an oncogene is measured by quantitating the level of a gene product, such as a nucleic acid (mRNA) or the translated protein encoded by the gene.

In an embodiment, the expression of the oncogene is measured at the protein level. Examples of methods to measure the amount/level of a protein in a sample include, but are not limited to: Western blot, immunoblot, enzyme-linked immunosorbent assay (ELISA), “sandwich” immunoassays, radioimmunoassay (RIA), immunoprecipitation, surface plasmon resonance (SPR), chemiluminescence, fluorescent polarization, phosphorescence, immunohistochemical (IHC) analysis, matrix-assisted laser desorption/ionization time-of-flight (MALDI-TOF) mass spectrometry, microcytometry, microarray, antibody array, microscopy (e.g., electron microscopy), flow cytometry, and proteomic-based assays.

In another embodiment, the expression of the oncogene is measured at the nucleic acid (mRNA, cDNA) level. Numerous detection and quantification technologies may be used to determine the expression level of the nucleic acids, including but not limited to: PCR, RT-PCR; RT-qPCR; NASBA; Northern blot technology; a hybridization array; branched nucleic acid amplification/technology; TMA; LCR; High-throughput sequencing or next generation sequencing (NGS) methods such as RNA-seq, in situ hybridization technology; and amplification process followed by HPLC detection or MALDI-TOF mass spectrometry. In a particular embodiment, an amplification process is performed by PCR.

In embodiments of the invention, all or part of a nucleic acid may be amplified and detected by methods such as the polymerase chain reaction (PCR) and variations thereof, such as, but not limited to, quantitative PCR (qPCR), reverse transcription PCR, and real-time PCR (including as a means of measuring the initial amounts of mRNA copies for each sequence in a sample). Such methods would utilize one or two primers that are complementary to portions of a nucleic acid, where the primers are used to prime nucleic acid synthesis. The newly synthesized nucleic acids are optionally labeled and may be detected directly or by hybridization to a polynucleotide of the invention. The newly synthesized nucleic acids may be contacted with polynucleotides (containing sequences) under conditions which allow for their hybridization. Additional methods to detect the expression of expressed nucleic acids include RNAse protection assays, including liquid phase hybridizations, and in situ hybridization of cells.

As would be understood by the skilled person, detection of expression of nucleic acids may be performed by the detection of expression of any appropriate portion or fragment of these nucleic acids, or the entire nucleic acids. Preferably, the portions are sufficiently large to contain unique sequences relative to other sequences expressed in a sample. Moreover, the skilled person would recognize that either strand of a nucleic acid may be detected as an indicator of expression of the nucleic acid. This follows because the nucleic acids are expressed as RNA molecules in cells, which may be converted to cDNA molecules for ease of manipulation and detection. The resultant cDNA molecules may have the sequences of the expressed RNA as well as those of the complementary strand thereto. Thus, either the RNA sequence strand or the complementary strand may be detected. Of course, it is also possible to detect the expressed RNA without conversion to cDNA.

In an embodiment, the method comprises performing a reverse transcription of mRNA molecules present in a sample; and amplifying the target cDNA and the one or more control cDNAs using primers hybridizing to the cDNAs.

The detection methods described herein are meant to exemplify how the present invention may be practiced and are not meant to limit the scope of invention. It is contemplated that other sequence-based methodologies for detecting the presence of a nucleic acid in a subject sample may be employed according to the invention.

### Chemotherapeutic Agents Used in Veterinary Oncology

In certain aspects, the present disclosure relates to the treatment of a cancer by administering to a subject at least one chemotherapeutic agent. Non-limiting examples of chemotherapeutic agents are listed in Table 1.

TABLE 1
Examples of chemotherapeutics and relevant indications
currently used in veterinary oncology

| Chemotherapeutic | Type of Cancer |
|---|---|
| Alkylating agents | |
| Cyclophosphamide | Lymphoma, mast |
| (Cytoxan) | cell tumors, |
| | mammary tumors, |
| | hemangiosarcomas |
| Ifosfamide (Ifex) | Chemoresistant |
| | lymphoma, soft |
| | tissue sarcoma |
| Chlorambucil (Leukeran) | Leukemias, mast |
| | cell tumors, |
| | lymphoma |
| Melphalan (Alkeran) | Multiple myeloma |
| Busulfan (Myeleran) | Leukemias |
| Procarbazine Hyd | Lymphoma |
| (Matulane) | |
| Plant alkaloids | |
| Vincristine (Oncovin) | Lymphoma, |
| | venereal tumors, |
| | mast cell tumors, |
| | sarcomas |
| Vinblastine | Lymphoma, mast |
| | cell tumors |
| Antimetabolites | |
| Methotrexate | Lymphoma, |
| | osteosarcoma |
| Cytosine arabinoside | CNS lymphoma, |
| (Cytostar, Ara-C) | leukemia |
| Fluorophyrimidines | Skin tumors, |
| (Fluorouracil [5-FU]) | mammary |
| | carcinoma, GI |
| | tumors |
| Hydroxyurea | Recurrent |
| | leukemias |
| Antitumor antibiotics | |
| Doxorabicin | Lymphoma, |
| (hydroxydaunomycin) | hemolymphatic |
| | malignancies, |
| | carcinomas and |
| | saracomas |
| | including |
| | osteosarcoma |
| Epirubicin (Pharmorubicin) | Lymphoma |
| Methoxymorpholino- | Chemoresistant |
| doxorubicon | lymphoma, |
| | sarcomas and |
| | carcinomas |
| Mitoxanthrone | Oral squamous |
| | cell carcinoma, |
| | lymphoma, |
| | sarcomas and |
| | carcinomas |
| Bleomycin | Squamous cell |
| | carcinoma |
| Actinomycin D | Lymphoma, |
| | sarcoma, |
| | carcinoma |
| Platinum compounds | |
| Cisplatin (Platinol) | Osteosarcoma, |
| | skin and nasal |
| | carcinomas |
| Carboplatin (Paraplatin) | Skin and nasal |
| | carcinomas |
| Lobaplatin | Osteosarcoma |
| Nitrosoureas | Brain and CNS |
| Lomustine | tumors, |
| | lymphomas, mast |
| | cell tumors |
| Carmustine | Brain tumors |
| Topoisomerase I inhibitors | |
| Camptothecins | Lymphoma |
| Hormones | |
| Prednisone | Lymphomas and |
| Biologic Response Modifiers | mast cell tumors |
| Peroxicam (Feldene) | Squamous cell |
| | carcinoma, |
| | mammary |
| | adenocarcinoma, |
| | transmissible |
| | venereal tumors |
| Muramyl dipeptide | Splenic |
| | hemangiosarcoma, |
| | osteosarcoma |
| Retinoids | |
| Etretinate (Tegison) | Cutaneous |
| Isotretinoin (Accutane) | lymphoma, |
| | mycosis fungoides |
| Other | |
| Paclitaxel (Taxol) | Mammary |
| | carcinomas, |
| | lymphomas |
| Darcarbazine | Recurrent |
| | lymphoma, |
| | melanoma, |
| | sarcomas |
| L-asparaginase (Elspar) | Lymphoid |
| | malignancies, mast |
| | cell tumors |

### G4-Stabilizing Ligands As Cancer Therapeutics

G-quadruplex (G4) DNA structures are four-stranded secondary DNA structures that play important roles in regulating gene expression. In the human genome, it is estimated that G4 structures can form at over 700,000 positions, and they are over-represented in oncogenes and regulatory genes and are under-represented in housekeeping and tumor suppressor genes (Eddy and Maizels, 2006; Huppert and Balasubramanian, 2007). G4 structures are thus suggested to be promising chemotherapeutic targets. This is further supported by the high occurrence of G4 structures in the telomeres and by their ability to obstruct DNA replication and repair, which leads to activation of the DNA damage response pathway resulting in apoptosis of cancer cells (Shay and Bacchetti, 1997). Furthermore, cancer cells possess more G4 DNA structures compared to non-cancerous cells (Biffi et al., 2014), and clinical trials have been conducted with G4-stabilizing compounds for treatment of BRCA1/2-deficient tumors (Xu et al., 2017) and carcinoid and neuroendocrine tumors (Dry gin et al., 2009).

In some aspects, the present disclosure relates to the treatment of a cancer by administering to a subject one or more G4-stabilizing ligands or compounds. Non-limiting examples of G4-stabilizing ligands include CX-3543, CX-5461, telomestatin, BMSG-SH-3, MM41, BRACO-19, quarfloxin, CM03, and PDP.

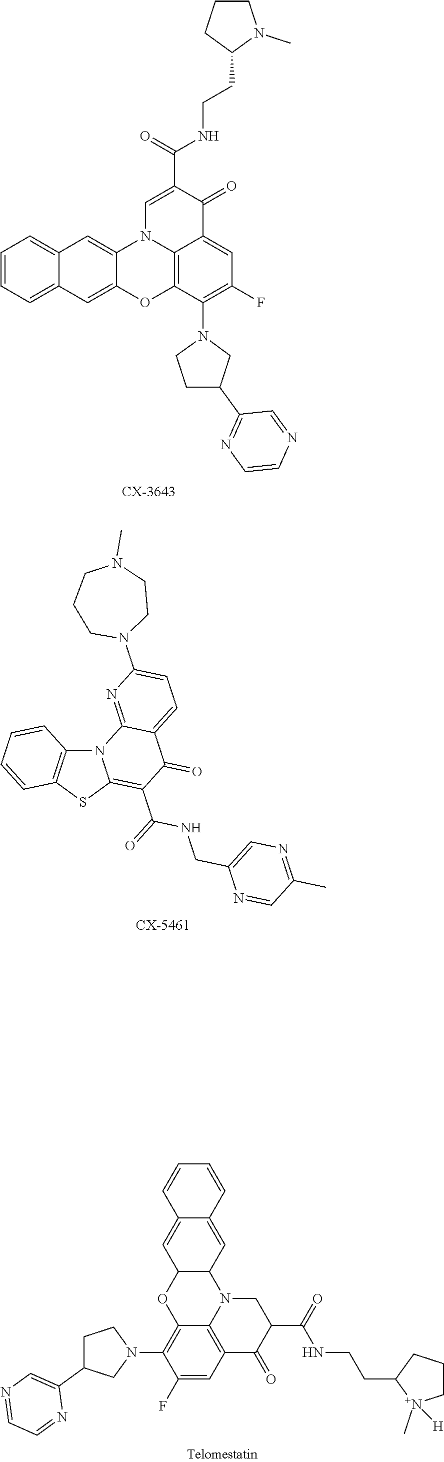 [SMILES: CN1CCC[C@H]1CCNC(=O)c1cn2c3c(c(N4CCC(c5cnccn5)C4)c(F)cc3c1=O)Oc1cc3ccccc3cc1-2 ; Cc1cnc(CNC(=O)c2c(=O)c3ccc(N4CCCN(C)CC4)nc3n3c2sc2ccccc23)cn1 ; C[NH+]1CCCC1CCNC(=O)C1CN2c3c(cc(F)c(N4CCC(c5cnccn5)C4)c3OC3C=c4ccccc4=CC32)C1=O]

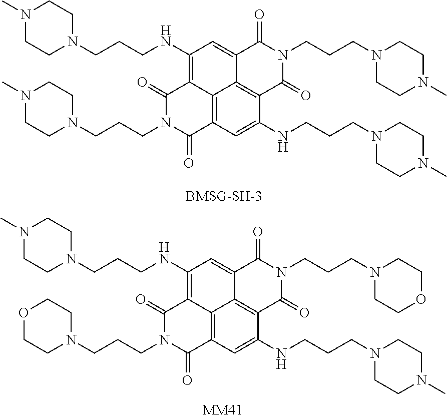

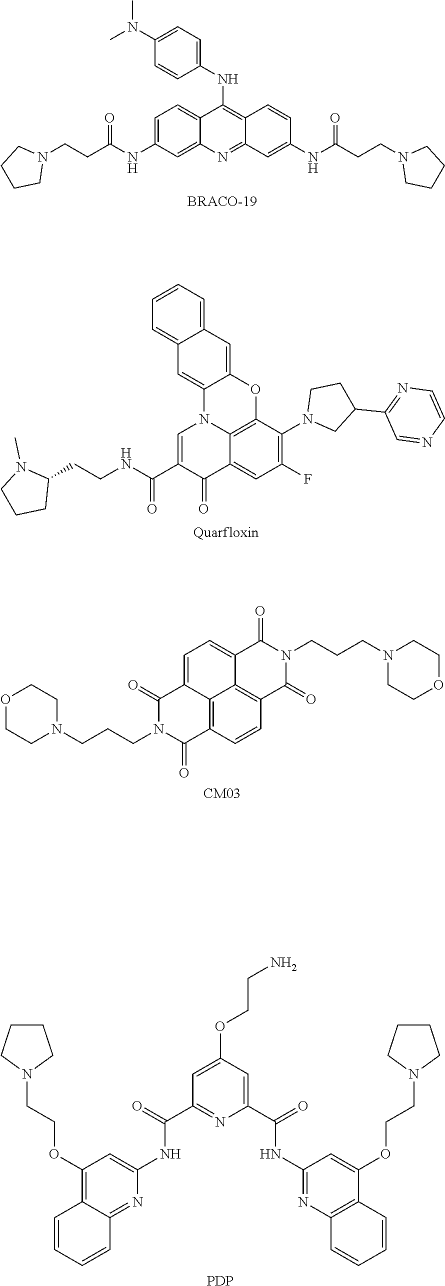

Additional non-limiting examples of G4-stabilizing ligands are shown in Table 2. See, also. Alessandrini I, et al., International Journal of Molecular Sciences. 2021; 22(11):5947; and Awadasseid, A. et al., Biomedicine & Pharmacotherapy. 2021; 139:111550.

TABLE 2
G4-stabilizing ligands

| Ligand | G4 Target | Tumor Models | Reference |
|---|---|---|---|
| Cisplatinum | Telomere | Osteosarcoma | Ju, H. -P. et al. ACS |
| derivatives | | | Omega 2016, 1, |
| (Tetra-Pt (bpy)) | | | 244-250 |
| Isoindoloquinoxalin | Telomere | Osteosarcoma | Salvati, E. et al., |
| derivatives | | | Oncogene 2010, 29, |
| | | | 6280-6293 |
| Antracene derivative | Telomere | Melanoma | Orlotti, N. I. et |
| (Ant1, 5) | | | al., Autophagy 2012, |
| | | | 8, 1185-1196 |
| Bisacridine | MYC | Squamous cell | Kuang, G. et al., J. |
| derivatives (a9) | | carcinoma | Med. Chem. 2020, 63, |
| | | | 9136-9153. |
| Benzofuran | MYC | Multiple | Gaikwad, S. M. et |
| derivative (D089) | | myeloma | al., Cancers 2020, |
| | | | 12, 2952 |
| Curcumin derivative | MYC | Cervical | Pandya, N. et al., |
| (Cur-4) | | carcinoma | Biochimie 2021, 180, |
| | | | 205-221. |
| 2,6-disubstituted | MYC | Cervical | Roy, S. et al., Eur. |
| amidoanthracene- | | carcinoma | J. Med. Chem. 2020, |
| 9,10-dione based | | | 195, 112202. |
| dimeric distamycin | | | |
| analogues (ANMP, | | | |
| ANDP and ANTP) | | | |
| Indenoisoquinolines | MYC | Breast cancer | Wang, K. B. et al., |
| | | | J. Am. Chem. Soc. |
| | | | 2019, 141, |
| | | | 11059-11070. |
| Functionalized | AR | Metastatic, | Tassinari, M. et al., |
| naphthalene diimide | | castration- | J. Med. Chem. 2018, |
| derivatives | | resistant | 61, 8625-8638. |
| (Compound 7) | | prostate cancer | |
| | | (mCRPC) | |
| Biimidazole | MYC | Acute myeloid | Hu, M. H. et al., |
| derivative (BIM-2) | BCL-2 | leukemia | Chem. 2020, 104, |
| | | | 104264. |
| Prolinamide-derived | MYC | Breast cancer | Paul, R. et al., Chem |
| peptidomimetic | BCL-2 | | Bio Chem 2020, 21, |
| (Ligand 1) | | | 963-970. |
| Core-extended | MYC | Melanoma | Recagni, M. et al., |
| naphthalene diimide | BCL-2 | | Cells 2019, 8, 1274. |
| derivatives | BRAF | | |
| | KIT | | |
| Imidazole-based | MYC | Metastatic | Zeng, L. et al., |
| tanshinone IIA | KRAS | triple-negative | Bioorg. Chem. 2021, |
| derivative | VEGF | Breast cancer | 106, 104433. |
| (Compound 4) | BCL-2 | | |
| | Telomere | | |
| Tetra-substituted | BCL-2 | Pancreatic | Ohnmacht, S. A. et |
| naphthalene-diimide | K-RAS | cancer | al., Sci. Rep. 2015, |
| derivative | | | 5, 11385 |
| (MM41) | | | |
| NDI derivative | KIT | Gastrointestinal | Gunaratnam, M. et |
| (Compound 1) | Telomere | stromal tumor | al., J. Med. Chem. |
| | | | 2009, 52, 3774-3783 |
| Symmetrical-and | EGFR | Metastatic, | Recagni, M. et al., |
| asymmetrical- | Telomere | castration- | Eur. J. Med. Chem. |
| substituted | | resistant | 2019, 177, 401-413 |
| naphthalene diimide | | prostate cancer | |
| derivatives | | (mCRPC) | |

The following examples are given for illustrative and non-limiting purposes of the present invention.

### EXAMPLES

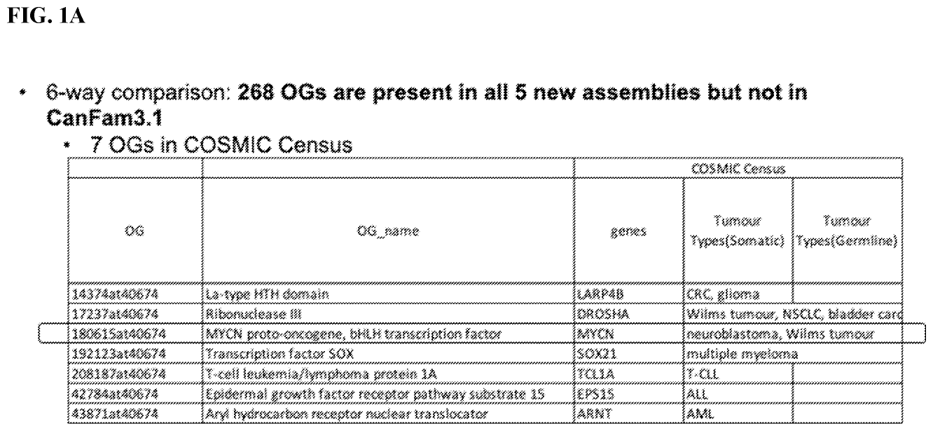

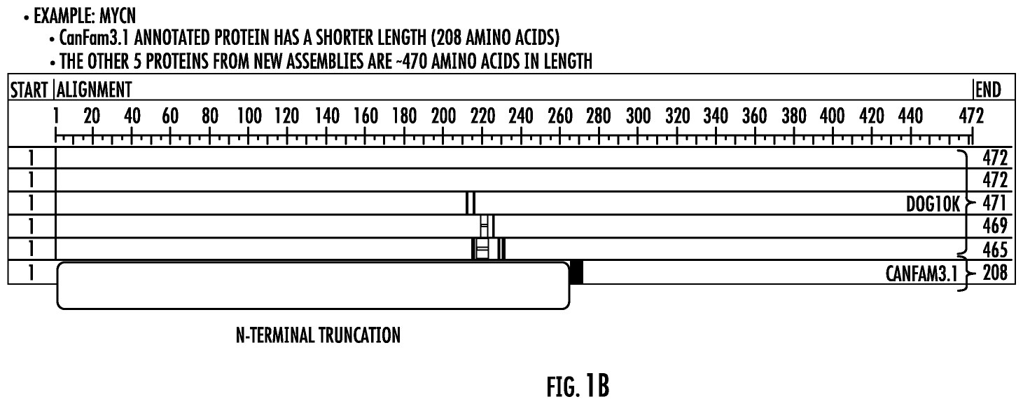

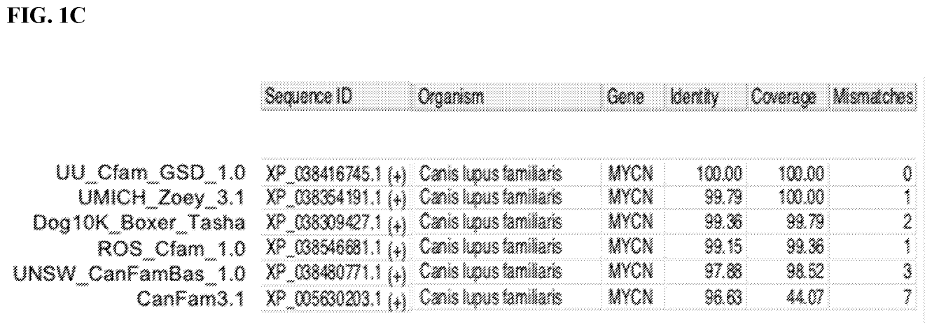

Example 1. Several Orthologs Are Missing in the CanFam3.1 Canine Genome Assembly Five recent canine genome assemblies (including the “Dog10K” assembly and others) generated with more advanced sequencing techniques were compared to an older canine genome assembly (designated herein as “CanFam3.1”). A six-way comparison of the canine genome assemblies revealed that 268 orthologous groups (OGs) were present in the five new genome assemblies but not in CanFam3.1. Among these 268 OGs, 7 OGs had corresponding human 10 orthologs in the Catalogue of Somatic Mutations in Cancer (COSMIC) Census (i.e., LARP4B, DROSHA, MYCN, SOX21, TCL1A, EPS15, and ARNT) giving rise to glioma, colorectal cancer (CRC), bladder cancer, kidney cancer (i.e., Wilms tumor), non-small cell lung cancer (NSCLC), neuroblastoma, multiple myeloma, T-cell chronic lymphocytic leukemia (T-CLL), acute lymphocytic leukemia (ALL), and acute myeloid leukemia (AML) (see FIG. 1A).

### Example 2. The MYCN Protein Sequence Shows an N-Terminal Truncation in the CanFam3.1 Canine Genome Assembly Arising from a 1.2 kb Assembly Gap

An alignment of the protein sequence for MYCN from five recent canine genome assemblies including the Dog10K canine genome assembly and the CanFam3.1 canine genome assembly was performed. The Dog10K assembly is a high-quality improved version of the same canine boxer individual (Tasha) which was used to generate CanFam3.1. This alignment revealed that while the MYCN protein from Dog10K was about 470 amino acids long, the MYCN protein from CanFam3.1 was only 208 amino acids long. The missing amino acids in the MYCN protein sequence from CanFam3.1 result in a significant N-terminal truncation (see FIGS. 1B and 1C).

A close inspection of the CanFam3.1 canine genome assembly identified a 1.2 kb assembly gap as responsible for this N-terminal truncation. The recently released canine reference genome assembly (i.e., Dog10K_Boxer_Tasha/canFam6) did not have this assembly gap.

### Example 3. The 1.2 kb Assembly Gap in MYCN is in a Highly G-Rich Region Typical of a G4 Structure

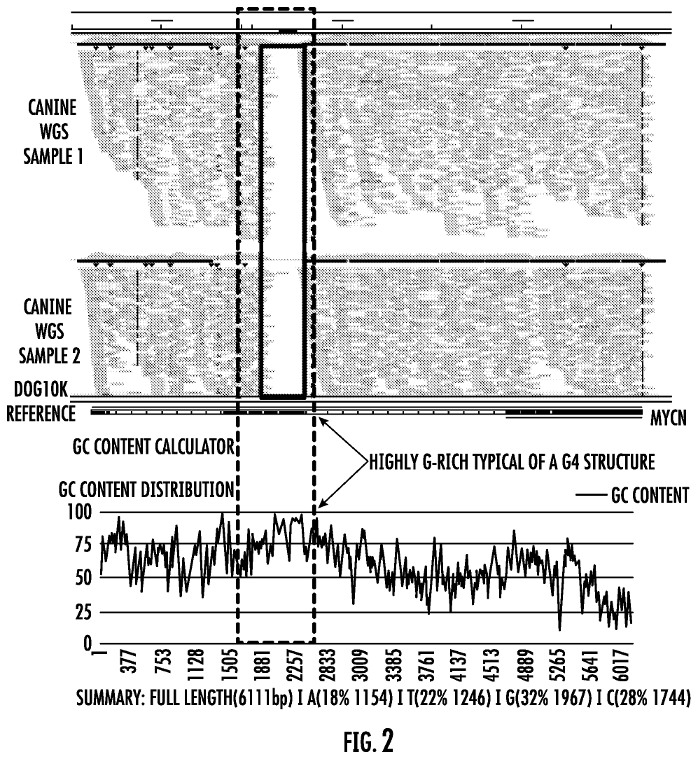

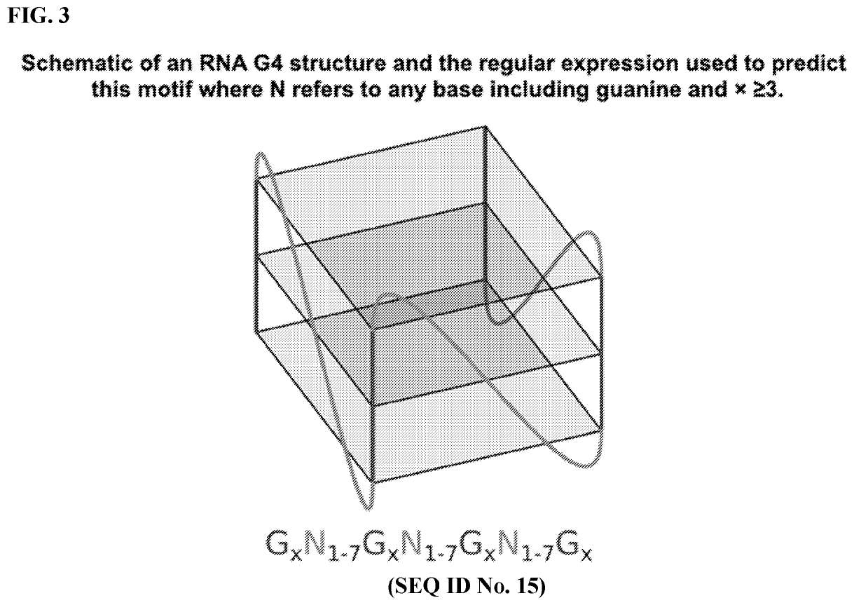

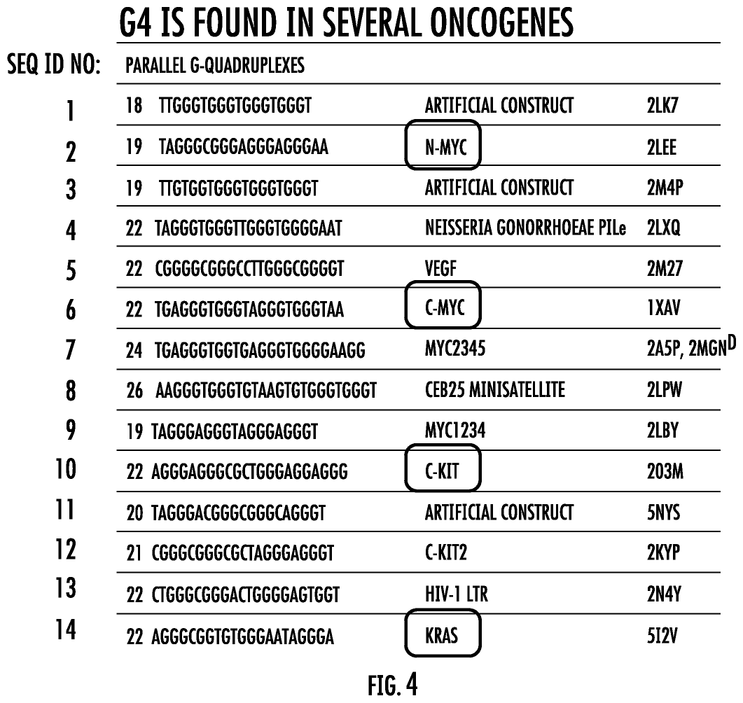

Further analysis of the 1.2 kb gap in MYCN comparing it to the corresponding sequence in the Dog10K reference showed the region to be highly G-rich, which is typical of a G4 structure (see FIG. 2). The G4 motif is identified by the expression of GₓN₁₋₇GₓN₁₋₇GₓN₁₋₇GₓN₁₋₇ (SEQ ID NO: 15) where N refers to any base including guanine and x≥3 (see FIG. 3). G4 structures have been implicated in several human oncogenes including N-myc, c-MYC, c-kit, and KRAS (see FIG. 4 with SEQ ID Nos 1-14 and Ghosh, A. et al., (2021) Nucleic acids research 49 (4): 2333-2345). Identification of the G-rich region typical of a G4 structure implicates G4-stabilizing ligands as chemotherapeutic agents in the treatment of cancers arising from mutations in the MYCN oncogene in canines.

### Example 4. The 1.2 kb Assembly Gap in Canine MYCN Spans the P44L/H Cancer Hotspot in Human MYCN

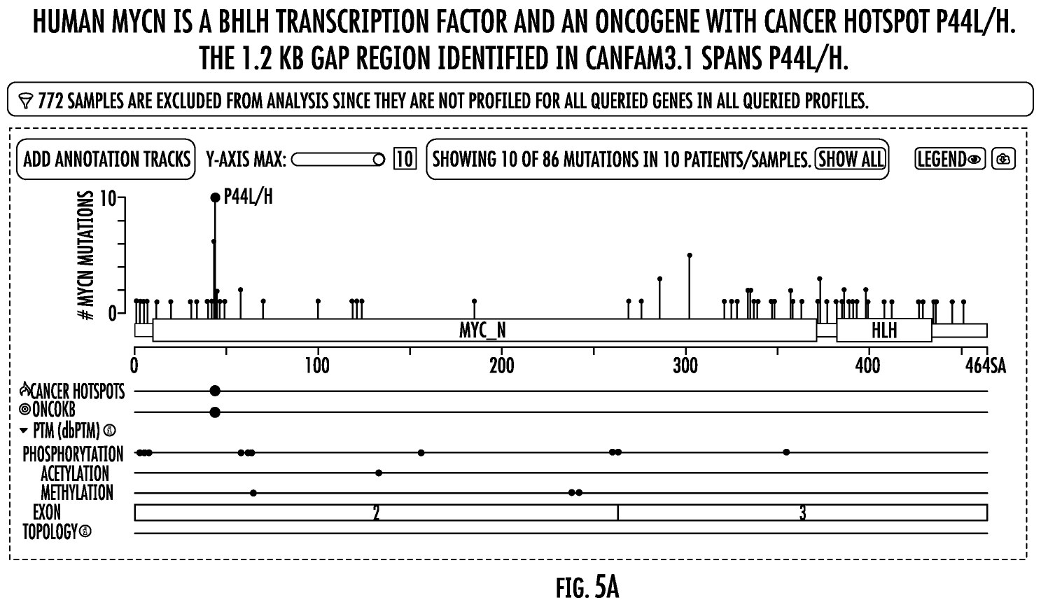

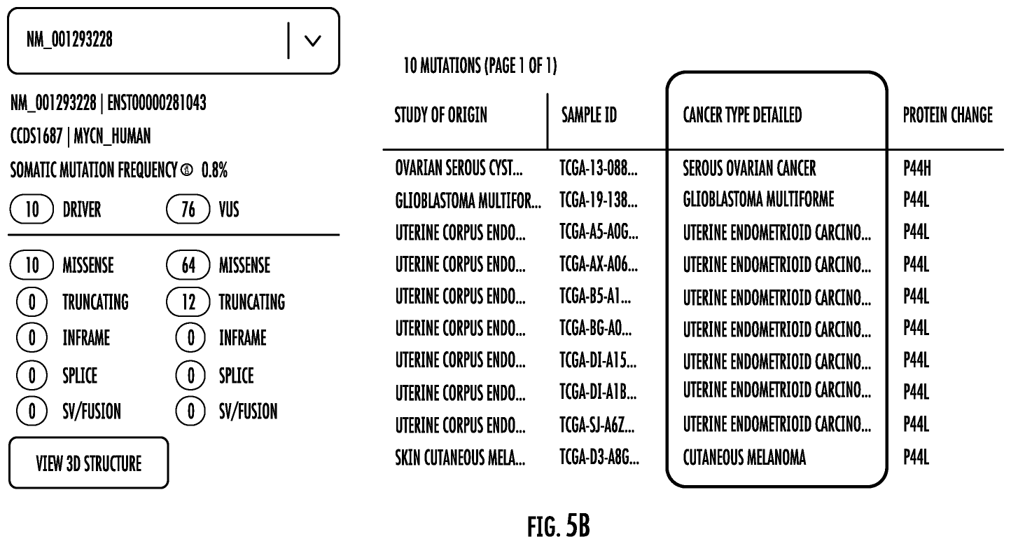

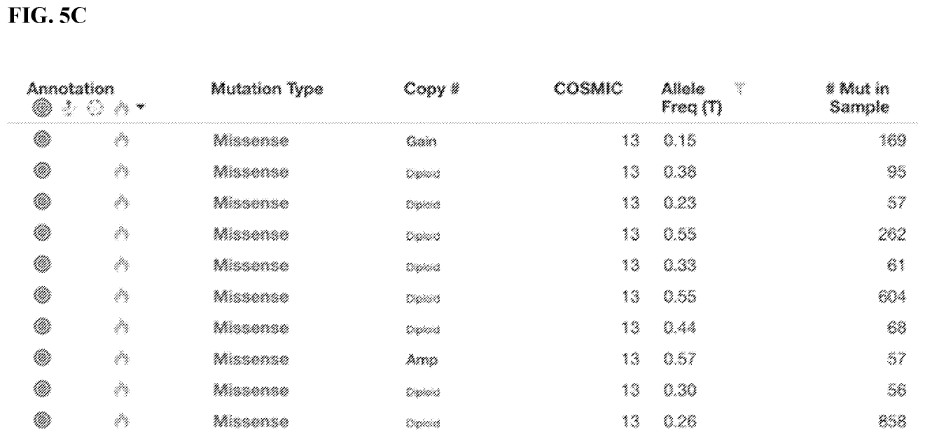

A comparison of the region absent in canine MYCN due to the 1.2 kb assembly gap with the corresponding region in human MYCN demonstrated that the assembly gap spans a prominent cancer hotspot known as P44L/H (see FIG. 5A). Mutations in the P44L/H cancer hotspot are present in human subjects suffering from ovarian cancer, uterine cancer, melanoma, and glioblastoma multiforme (see FIGS. 5B and 5C).

### Example 5. Pattern Matching Predicts a G4 Motif in the 1.2 kb Gap G-Rich Region Missing From the CanFam3.1 Assembly

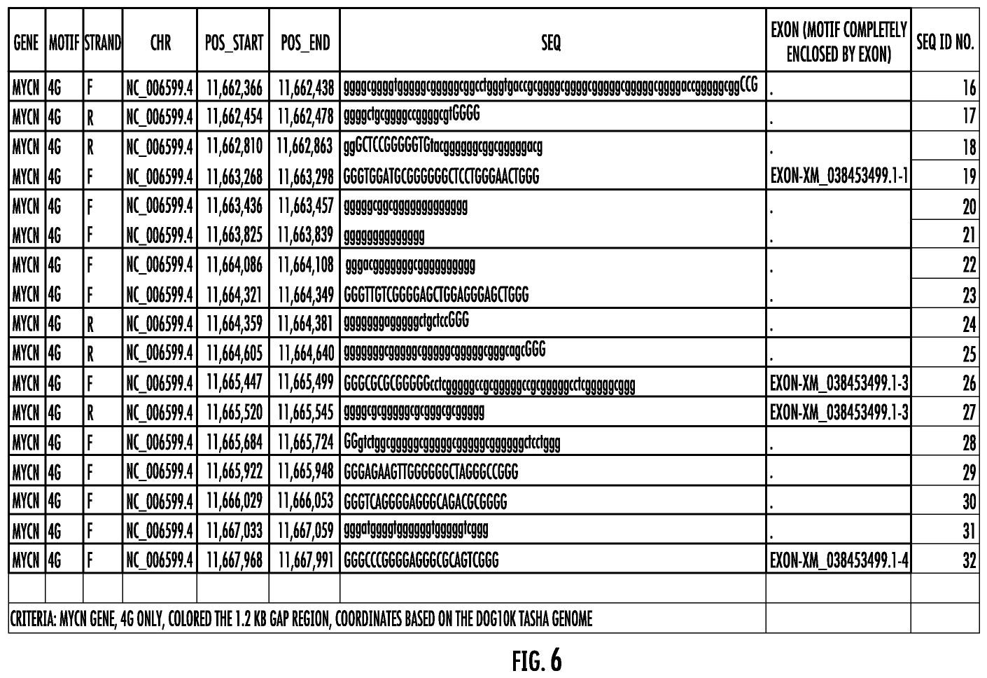

Using the methods described in Gong J Y, et al. Proc Natl Acad Sci USA. 2021; 118 (21): e2013230118, pattern matching was used to predict G4 motifs along the canine MYCN gene. Several G4 motifs were predicted along the canine MYCN gene. Importantly, a predicted G4 motif was found within the 1.2 kb gap G-rich region which is missing from the CanFam3.1 assembly (see FIG. 6, SEQ ID Nos. 16-32).

All headings are for the convenience of the reader and should not be used to limit the meaning of the text that follows the heading, unless so specified.

Unless defined otherwise, all technical and scientific terms herein have the same meaning as commonly understood by one of ordinary skill in the art to which this invention belongs. Although any methods and materials, similar or equivalent to those described herein, can be used in the practice or testing of the present invention, the preferred methods and materials are described herein. All publications, patents, and patent publications cited are incorporated by reference herein in their entirety for all purposes.

The publications discussed herein are provided solely for their disclosure prior to the filing date of the present application. Nothing herein is to be construed as an admission that the present invention is not entitled to antedate such publication by virtue of prior invention.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth.

1. A method of selecting a subject for a G4-stabilizing ligand cancer treatment, the method comprising:
identifying if an oncogene comprising a G4 motif comprising GₓN₁₋₇GₓN₁₋₇GₓN₁₋₇GₓN₁₋₇ (SEQ ID NO: 15) where N refers to any base and x≥3 is expressed in the subject's genome;
determining an expression level of the subject's oncogene; and
selecting the subject for the G4-stabilizing ligand cancer treatment if an increase in the level of expression of the oncogene at least 50% higher than that of non-cancerous cells that are adjacent to the cancer is detected.

2. The method of claim 1, wherein the expression level of the oncogene is determined at the protein level by Western blotting, ELISA-based detection, in situ immunohistochemistry, in situ immunocytochemistry, affinity chromatography, or an enzyme immunoassay.

3. The method of claim 1, wherein the expression level of the oncogene is determined at the nucleic acid level by Northern blotting, real time PCR, RT PCR, quantitative PCR, RT-qPCR, a hybridization array, branched nucleic acid amplification, RNA-seq, in situ hybridization, amplification followed by HPLC detection or MALDI-TOF mass spectrometry, or next generation sequencing (NGS).

4. The method of claim 1,
further comprising administering the G4-stabilizing ligand to the subject.

5. The method of claim 1,
wherein the oncogene is MYCN; and the cancer cell is derived from a cancer selected from the group consisting of: glioma, colorectal cancer (CRC), bladder cancer, kidney cancer, non-small cell lung cancer (NSCLC), neuroblastoma, multiple myeloma, T-cell chronic lymphocytic leukemia (T-CLL), acute lymphocytic leukemia (ALL), acute myeloid leukemia (AML), ovarian cancer, uterine cancer, melanoma, and glioblastoma multiforme.

6. (canceled)

7. The method of claim 5, wherein the cancer is selected from the group consisting of ovarian cancer, uterine cancer, melanoma, and glioblastoma multiforme; and the G4-stabilizing ligand is selected from the group consisting of CX-3543, CX-5461, telomestatin, BMSG-SH-3, MM41, BRACO-19, quarfloxin, CM03, PDP, and pharmaceutically acceptable salts thereof.

8. (canceled)

9. The method of claim 1,
further comprising administering to the subject a chemotherapeutic agent selected from the group consisting of an alkylating agent, plant alkaloid, antimetabolite, antitumor antibiotic, platinum compound, topoisomerase I inhibitor, retinoid, and combinations thereof.

10. The method of claim 1,
wherein the subject is a canine.

11. A method of detecting one or more cancer cells susceptible to growth inhibition with a G4-stabilizing ligand, the method comprising:
identifying an oncogene in the genome of the one or more cancer cells having a G4 motif comprising GₓN₁₋₇GₓN₁₋₇GₓN₁₋₇GₓN₁₋₇ (SEQ ID NO: 15) where N refers to any base and x≥3;
determining an expression level of the oncogene in the one or more cancer cells; and
identifying cancer cells having an increase in the level of expression of the oncogene at least 50% higher than that of non-cancerous cells that are adjacent to the cancer cells as susceptible to growth inhibition with the G4-stabilizing ligand.

12. The method of claim 11, wherein the oncogene is MYCN; and the cancer cell is derived from a cancer selected from the group consisting of: glioma, colorectal cancer (CRC), bladder cancer, kidney cancer, non-small cell lung cancer (NSCLC), neuroblastoma, multiple myeloma, T-cell chronic lymphocytic leukemia (T-CLL), acute lymphocytic leukemia (ALL), acute myeloid leukemia (AML), ovarian cancer, uterine cancer, melanoma, and glioblastoma multiforme.

13. (canceled)

14. The method of claim 11, further comprising administering a G4-stabilizing ligand to the cancer cell to confirm susceptibility to growth inhibition; and the G4-stabilizing ligand is selected from the group consisting of CX-3543, CX-5461, telomestatin, BMSG-SH-3, MM41, BRACO-19, quarfloxin, CM03, PDP, and pharmaceutically acceptable salts thereof.

15. (canceled)

16. (canceled)

17. The method of claim 11, wherein
the expression level of the oncogene is determined at the protein level by Western blotting, ELISA-based detection, in situ immunohistochemistry, in situ immunocytochemistry, affinity chromatography, or an enzyme immunoassay; and/or
the expression level of the oncogene is determined at the nucleic acid level by Northern blotting, real time PCR, RT PCR, quantitative PCR, RT-qPCR, a hybridization array, branched nucleic acid amplification, RNA-seq, in situ hybridization, amplification followed by HPLC detection or MALDI-TOF mass spectrometry, or next generation sequencing (NGS).

18. A method of treating a subject having cancer where an oncogene comprising a G4 motif comprising GₓN₁₋₇GₓN₁₋₇GₓN₁₋₇GₓN₁₋₇ (SEQ ID NO: 15) where N refers to any base and x≥3 is expressed in the subject's genome, the method comprising administering a G4-stabilizing ligand to the subject.

19. The method of claim 18, additionally comprising identifying an oncogene expressed in the genome of the subject with a G4 motif comprising GₓN₁₋₇ GₓN₁₋₇GₓN₁₋₇GₓN₁₋₇ (SEQ ID NO: 15) where N refers to any base and x≥3; and
determining an expression level of the oncogene in the subject, wherein an increase in expression level of at least 50% higher than that of non-cancerous cells that are adjacent to the cancer identifies the subject for treatment with a G4-stabilizing ligand.

20. The method of claim 19, wherein the expression level of the oncogene is determined at the protein level by Western blotting, ELISA-based detection, in situ immunohistochemistry, in situ immunocytochemistry, affinity chromatography, or an enzyme immunoassay.

21. The method of claim 19, wherein the expression level of the oncogene is determined at the nucleic acid level by Northern blotting, real time PCR, RT PCR, quantitative PCR, RT-qPCR, a hybridization array, branched nucleic acid amplification, RNA-seq, in situ hybridization, amplification followed by HPLC detection or MALDI-TOF mass spectrometry, or next generation sequencing (NGS).

22. The method of claim 18, wherein the oncogene is MYCN; and the cancer is selected from the group consisting of glioma, colorectal cancer (CRC), bladder cancer, kidney cancer, non-small cell lung cancer (NSCLC), neuroblastoma, multiple myeloma, T-cell chronic lymphocytic leukemia (T-CLL), acute lymphocytic leukemia (ALL), acute myeloid leukemia (AML), ovarian cancer, uterine cancer, melanoma, and glioblastoma multiforme.

23. (canceled)

24. The method of claim 22, wherein the cancer is selected from the group consisting of ovarian cancer, uterine cancer, melanoma, and glioblastoma multiforme; and the G4-stabilizing ligand is selected from the group consisting of: CX-3543, CX-5461, telomestatin, BMSG-SH-3, MM41, BRACO-19, quarfloxin, CM03, PDP, and pharmaceutically acceptable salts thereof.

25. (canceled)

26. The method of claim 18, further comprising administering to the subject a chemotherapeutic agent selected from the group consisting of an alkylating agent, plant alkaloid, antimetabolite, antitumor antibiotic, platinum compound, topoisomerase I inhibitor, retinoid, and combinations thereof.

27. The method of claim 18, wherein the subject is a canine.


SEQUENCE LISTING (32 sequences)

SEQ ID NO: 1 - DNA, 18 bp, synthetic construct
  source 1..18: mol_type = other DNA
ttgggtgggtgggtgggt

SEQ ID NO: 2 - DNA, 19 bp, Homo sapiens
  source 1..19: mol_type = genomic DNA
tagggcgggagggagggaa

SEQ ID NO: 3 - DNA, 19 bp, synthetic construct
  source 1..19: mol_type = other DNA
ttgtggtgggtgggtgggt

SEQ ID NO: 4 - DNA, 22 bp, Neisseria gonorrhoeae
  source 1..22: mol_type = genomic DNA
tagggtgggttgggtggggaat

SEQ ID NO: 5 - DNA, 22 bp, Homo sapiens
  source 1..22: mol_type = genomic DNA
cggggcgggccttgggcggggt

SEQ ID NO: 6 - DNA, 22 bp, Homo sapiens
  source 1..22: mol_type = genomic DNA
tgagggtgggtagggtgggtaa

SEQ ID NO: 7 - DNA, 24 bp, Homo sapiens
  source 1..24: mol_type = genomic DNA
tgagggtggtgagggtggggaagg

SEQ ID NO: 8 - DNA, 26 bp, Homo sapiens
  source 1..26: mol_type = genomic DNA
aagggtgggtgtaagtgtgggtgggt

SEQ ID NO: 9 - DNA, 19 bp, Homo sapiens
  source 1..19: mol_type = genomic DNA
tagggagggtagggagggt

SEQ ID NO: 10 - DNA, 22 bp, Homo sapiens
  source 1..22: mol_type = genomic DNA
agggagggcgctgggaggaggg

SEQ ID NO: 11 - DNA, 20 bp, synthetic construct
  source 1..20: mol_type = other DNA
tagggacgggcgggcagggt

SEQ ID NO: 12 - DNA, 21 bp, Homo sapiens
  source 1..21: mol_type = genomic DNA
cgggcgggcgctagggagggt

SEQ ID NO: 13 - DNA, 22 bp, Human immunodeficiency virus 1
  source 1..22: mol_type = genomic DNA
ctgggcgggactggggagtggt

SEQ ID NO: 14 - DNA, 22 bp, Homo sapiens
  source 1..22: mol_type = genomic DNA
agggcggtgtgggaatagggaa

SEQ ID NO: 15 - DNA, 40 bp, synthetic construct
  source 1..40: mol_type = other DNA
  misc_difference 1..3: note = A first guanine-rich region, wherein the guanine-rich region comprises at least three guanine residues
  misc_difference 11..13: note = A second guanine-rich region, wherein the guanine-rich region comprises at least three guanine residues
  misc_difference 21..23: note = A third guanine-rich region, wherein the guanine-rich region comprises at least three guanine residues
  misc_difference 31..33: note = A fourth guanine-rich region, wherein the guanine-rich region comprises at least three guanine residues
  misc_difference 4..10: replace = n
  misc_difference 4..10: replace = nn
  misc_difference 4..10: replace = nnn
  misc_difference 4..10: replace = nnnn
  misc_difference 4..10: replace = nnnnn
  misc_difference 4..10: replace = nnnnnn
  misc_difference 14..20: replace = n
  misc_difference 14..20: replace = nn
  misc_difference 14..20: replace = nnn
  misc_difference 14..20: replace = nnnn
  misc_difference 14..20: replace = nnnnn
  misc_difference 14..20: replace = nnnnnn
  misc_difference 24..30: replace = n
  misc_difference 24..30: replace = nn
  misc_difference 24..30: replace = nnn
  misc_difference 24..30: replace = nnnn
  misc_difference 24..30: replace = nnnnn
  misc_difference 24..30: replace = nnnnnn
  misc_difference 34..40: replace = n
  misc_difference 34..40: replace = nn
  misc_difference 34..40: replace = nnn
  misc_difference 34..40: replace = nnnn
  misc_difference 34..40: replace = nnnnn
  misc_difference 34..40: replace = nnnnnn
gggnnnnnnngggnnnnnnngggnnnnnnngggnnnnnnn

SEQ ID NO: 16 - DNA, 71 bp, Canis lupus
  source 1..71: mol_type = genomic DNA
ggggcggggtgggggcgggggcggcctgggtgaccgcggggcgggggcgggggcggggacgggggcggccg

SEQ ID NO: 17 - DNA, 25 bp, Canis lupus
  source 1..25: mol_type = genomic DNA
ggggctgcggggccggggcgtgggg

SEQ ID NO: 18 - DNA, 54 bp, Canis lupus
  source 1..54: mol_type = genomic DNA
gggctccgggggtgtgtacggggggcggcgggggacgggagggggtggcggggg

SEQ ID NO: 19 - DNA, 31 bp, Canis lupus
  source 1..31: mol_type = genomic DNA
gggtggatgcggggggctcctgggaactggg

SEQ ID NO: 20 - DNA, 22 bp, Canis lupus
  source 1..22: mol_type = genomic DNA
gggggcggcggggggggggggg

SEQ ID NO: 21 - DNA, 15 bp, Canis lupus
  source 1..15: mol_type = genomic DNA
ggggggggggggggg

SEQ ID NO: 22 - DNA, 23 bp, Canis lupus
  source 1..23: mol_type = genomic DNA
gggacgggggggcgggggggggg

SEQ ID NO: 23 - DNA, 29 bp, Canis lupus
  source 1..29: mol_type = genomic DNA
gggttgtcggggagctggagggagctggg

SEQ ID NO: 24 - DNA, 23 bp, Canis lupus
  source 1..23: mol_type = genomic DNA
gggggggagggggctgctccggg

SEQ ID NO: 25 - DNA, 36 bp, Canis lupus
  source 1..36: mol_type = genomic DNA
gggggggcgggggcgggggcgggggcgggcagcggg

SEQ ID NO: 26 - DNA, 53 bp, Canis lupus
  source 1..53: mol_type = genomic DNA
gggcgcgcgggggcctcgggggccgcgggggccgcgggggcctcgggggcggg

SEQ ID NO: 27 - DNA, 26 bp, Canis lupus
  source 1..26: mol_type = genomic DNA
ggggcgcgggggcgcgggcgcggggg

SEQ ID NO: 28 - DNA, 41 bp, Canis lupus
  source 1..41: mol_type = genomic DNA
gggtctggcgggggcgggggcgggggcggggggctcctggg

SEQ ID NO: 29 - DNA, 27 bp, Canis lupus
  source 1..27: mol_type = genomic DNA
gggagaagttggggggctagggccggg

SEQ ID NO: 30 - DNA, 25 bp, Canis lupus
  source 1..25: mol_type = genomic DNA
gggtcaggggagggcagacgcgggg

SEQ ID NO: 31 - DNA, 27 bp, Canis lupus
  source 1..27: mol_type = genomic DNA
gggatggggtggggggtgggggtcggg

SEQ ID NO: 32 - DNA, 24 bp, Canis lupus
  source 1..24: mol_type = genomic DNA
gggcccggggagggcgcagtcggg